(19) United States
(12) Patent Application Publication
(10) Pub. No.: US 2016/0018393 A1
(43) Pub. Date: Jan. 21, 2016

(54) TRANSMISSION-LINE-COUPLED MICROFLUIDIC-CHIP TECHNOLOGY FOR ELECTROMAGNETIC SENSING OF BIOMOLECULES AND BIOPARTICLES

(71) Applicants: Elliott R. Brown, Beavercreek, OH (US); Leamon Viveros, Beavercreek, OH (US); Edgar A. Mendoza, Redondo Beach, CA (US)

(72) Inventors: Elliott R. Brown, Beavercreek, OH (US); Leamon Viveros, Beavercreek, OH (US); Edgar A. Mendoza, Redondo Beach, CA (US)

(73) Assignee: WRIGHT STATE UNIVERSITY, Dayton, OH (US)

(21) Appl. No.: 14/802,116

(22) Filed: Jul. 17, 2015

Related U.S. Application Data
(60) Provisional application No. 62/025,736, filed on Jul. 17, 2014.

(51) Int. Cl. G01N 33/543; B01L 3/00; H01P 3/00

(52) U.S. Cl. CPC G01N 33/54373; H01P 3/003; B01L 3/502715; B01L 3/502761; G01N 2469/00; B01L 2200/0647; B01L 2300/0636; B01L 2300/12; B01L 2300/0861

(57) ABSTRACT

A coplanar waveguide transmission line for use in detecting biomolecules and bioparticles is provided that includes a signal conductor disposed on a top surface of the dielectric substrate, a ground conductor disposed on the top surface of the dielectric substrate on each side of the signal conductor, a continuous gap defined between the signal conductor and each of the ground conductors, micro-channels disposed below a top surface of the dielectric substrate, and reservoirs disposed below the top surface of the substrate.

### CROSS-REFERENCE TO RELATED APPLICATIONS

This application claims the benefit of U.S. Provisional Patent Application Ser. No. 62/025,736 entitled “A TRANSMISSION-LINE-COUPLED MICRO-FLUIDIC-CHIP TECHNOLOGY FOR ELECTROMAGNETIC SENSING OF BIOMOLECULES AND BIOPARTICLES” filed on Jul. 17, 2014. The entirety of the above-noted application is incorporated herein by reference.

### ORIGIN

The innovation disclosed herein relates to microfluidic-chip technology and more specifically, microfluidic-chip technology for electromagnetic sensing of biomolecules and bioparticles.

### BACKGROUND

Due to their large mass and “soft” hydrogen bonds, biomolecules and bioparticles can vibrate resonantly at much lower frequencies than smaller molecules, sometimes in the THz region of the electromagnetic spectrum. The challenge in measuring these resonances by radiative spectroscopy is their quality factor (Q) and their polarity. Because of the high absorption by liquid water at all frequencies below the infrared radiation waves, there is strong background attenuation in any aqueous biosample, which can mask the biomolecular resonance. Finally, assuming a resonant vibration with suitably weak damping, the molecule still has to have a net dipole moment (i.e., polarity) to interact with radiation. Some biomolecules (e.g., nucleic acids) have strong polarity in their physiological state, whereas others (e.g., polysaccharides) are less polar.

In measuring resonant signatures, sample preparation and presentation is very important, as is instrumentation. The samples to be detected often have very small volume or, if the volume is substantial, have very low biomolecular concentration. Therefore, it is very important that the sample holder operate with small biosample volumes and very efficient interaction between this volume and the interrogating electromagnetic radiation.

In addition, during the past several years, there has been an explosion of improvements in respiratory virus diagnostics, from novel specimen collection instruments to highly sensitive and multiplexed nucleic acid amplification tests. With the expanding list of antigen and molecular-based tests, it is now possible for laboratories to offer comprehensive testing for respiratory viruses without even performing virus isolation. Recent advances have also been made to this traditional approach to improve the turn-around time by using a combination of shell vial cultures and immunostaining.

Because of the sensitivity and rapid turn-around time, nucleic acid amplification technologies such as polymerase chain reaction (PCR) will likely be the focus of further advances in respiratory virus diagnostics. The future will likely promise additional commercial test kits for molecular detection of respiratory viruses, including multiplexed assays. Because of their exquisite sensitivity, nucleic acid amplification tests can create diagnostic conundrums. Additional research is needed to elucidate the clinical significance of positive PCR results that are persistent in a patient, and when two or more respiratory viruses are detected in a single specimen.

Future research on respiratory virus diagnostics should aim towards the ability to accurately detect a spectrum of clinically significant viruses rapidly enough to affect patient management and initiation of infection control measures while keeping the costs affordable. Commercial kits provide standardization not achievable with laboratory-developed tests, but at a substantially higher cost. Ultimately, the utilization of molecular testing, particularly highly multiplexed tests in routine patient management will depend on the cost/benefit ratio. Current and future diagnostic options will include antigen, molecular, and culture-based methods. The performance characteristics and limitations of these methods will vary greatly with the new generations of assays.

At present, the detection of viruses in human beings is carried out by indirect means, such as the measurement of antibodies in the blood, or by PCR. Antibodies are produced by the human body in response to viral maladies, and usually take the form of specific proteins. PCR is a biochemical technology in molecular biology used to amplify a single or a few copies of a piece of DNA across several orders of magnitude, generating thousands to millions of copies of a particular DNA sequence. In both cases there are problems with false positives.

### SUMMARY

The following presents a simplified summary in order to provide a basic understanding of some aspects of the innovation. This summary is not an extensive overview of the innovation. It is not intended to identify key/critical elements or to delineate the scope of the innovation. Its sole purpose is to present some concepts of the innovation in a simplified form as a prelude to the more detailed description that is presented later.

In an aspect of the innovation, a coplanar waveguide transmission line for use in detecting biomolecules and bioparticles is disclosed that includes a dielectric substrate, a signal conductor disposed on a top surface of the dielectric substrate, a ground conductor disposed on the top surface of the dielectric substrate on each side of the signal conductor, a continuous gap defined between the signal conductor and each of the ground conductors, a plurality of micro-channels disposed below a top surface of the dielectric substrate, and a plurality of reservoirs disposed below the top surface of the substrate, wherein the plurality of reservoirs supply an aqueous solution to the micro-channels.

In another aspect of the innovation a microfluidic bio-detection chip is disclosed that includes a dielectric substrate, a coplanar waveguide transmission line disposed on a top surface of the dielectric substrate, the coplanar waveguide transmission line including, a pad region, a microfluidic region, and a taper region connecting the pad region to the microfluidic region, a pair of continuous gaps extending along the coplanar waveguide transmission line, a microchannel region disposed below a top surface of the dielectric substrate, and a plurality of reservoirs disposed below the top surface of the substrate, wherein the plurality of reservoirs supply an aqueous solution to the microchannel region.

In yet another aspect of the innovation a method of detecting a virus is disclosed that includes providing a coplanar waveguide transmission line having a pad section, a taper section, and a microfluidic section, the microfluidic section being disposed above a microchannel region, generating an electric field in a pair of continuous gaps defined in the coplanar waveguide transmission line, coupling via the electric field the microfluidic region and the microchannel region, monitoring for a presence of an electromagnetic signature in a biosample, and detecting a presence of biomolecules and bioparticles.

To accomplish the foregoing and related ends, certain illustrative aspects of the innovation are described herein in connection with the following description and the annexed drawings. These aspects are indicative, however, of but a few of the various ways in which the principles of the innovation can be employed and the subject innovation is intended to include all such aspects and their equivalents. Other advantages and novel features of the innovation will become apparent from the following detailed description of the innovation when considered in conjunction with the drawings.

### BRIEF DESCRIPTION OF THE DRAWINGS

FIG. 1 is a transmittance vs. frequency graph of radiative power through an innovative microfluidic bio-detection chip in accordance with the innovation.

FIG. 2 is a top view of the innovative microfluidic bio-detection chip in accordance with the innovation.

FIG. 3 is a close up view of a lower half of the microfluidic bio-detection chip shown in FIG. 2 in accordance with an aspect of the innovation.

FIG. 4 is a CPW transmission line showing a plane of antisymmetry in accordance with an aspect of the innovation.

FIG. 5 is a side view of a microchannel region in a cut taken vertically down an axis of propagation and through a center of a signal conductor in accordance with an aspect of the innovation.

FIG. 6 is a graph of real and imaginary parts of a dielectric function of a pure aqueous solution in accordance with an aspect of the innovation.

FIG. 7 is a graph of real and imaginary parts of a dielectric function of an aqueous solution mixture in accordance with an aspect of the innovation.

FIG. 8 is a graph of an attenuation coefficient of the pure aqueous solution and the mixture in accordance with an aspect of the innovation.

FIG. 9 is a graph of the attenuation length of the pure aqueous solution and the mixture in accordance with an aspect of the innovation.

FIG. 10 is another example embodiment of an innovative microfluidic bio-detection chip in accordance with an aspect of the innovation.

FIG. 11 is an end view of the microfluidic chip of FIG. 10 illustrating a coplanar waveguide disposed between a substrate and a superstrate in accordance with an aspect of the innovation.

FIG. 12 is a block diagram illustrating a method of detecting a virus in accordance with an aspect of the innovation.

### DETAILED DESCRIPTION

The innovation is now described with reference to the drawings, wherein like reference numerals are used to refer to like elements throughout. In the following description, for purposes of explanation, numerous specific details are set forth in order to provide a thorough understanding of the subject innovation. It may be evident, however, that the innovation can be practiced without these specific details. In other instances, well-known structures and devices are shown in block diagram form in order to facilitate describing the innovation.

While specific characteristics are described herein (e.g., thickness), it is to be understood that the features, functions and benefits of the innovation can employ characteristics that vary from those described herein. These alternatives are to be included within the scope of the innovation and claims appended hereto.

While, for purposes of simplicity of explanation, the one or more methodologies shown herein, e.g., in the form of a flow chart, are shown and described as a series of acts, it is to be understood and appreciated that the subject innovation is not limited by the order of acts, as some acts may, in accordance with the innovation, occur in a different order and/or concurrently with other acts from that shown and described herein. For example, those skilled in the art will understand and appreciate that a methodology could alternatively be represented as a series of interrelated states or events, such as in a state diagram. Moreover, not all illustrated acts may be required to implement a methodology in accordance with the innovation.

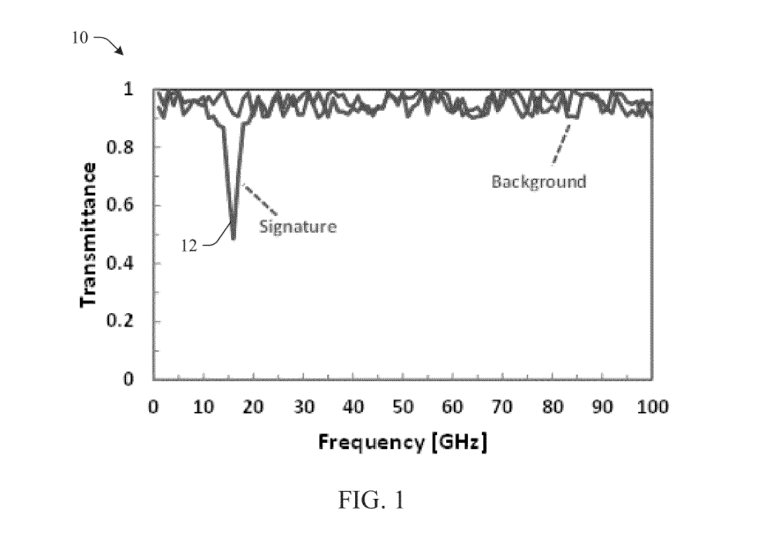

Disclosed herein is an innovative microfluidic bio-detection chip used to detect biomolecules and bioparticles in accordance with an aspect of the innovation. The innovation determines the presence of biomolecules or particles, such as proteins, nucleic acids, cytokines, viruses, and cells, by their unique electromagnetic signature. More specifically, as shown in the transmittance vs frequency graph 10 in FIG. 1, a dip 12 in the transmission spectrum of radiative power through the microfluidic bio-detection chip at a unique frequency indicates the presence of biomolecules or bio particles. The dip 12 is narrow enough in frequency to be clearly discernible from other transmission features, such as the instrumental noise shown in background scan or the noise away from the resonance signature. The radiation can be scanned in frequency by a calibrated sweep oscillator that can operate up to 100 GHz or higher. When combined with a sensitive receiver and modulation scheme, this technology will provide a dynamic range of 80 dB or higher.

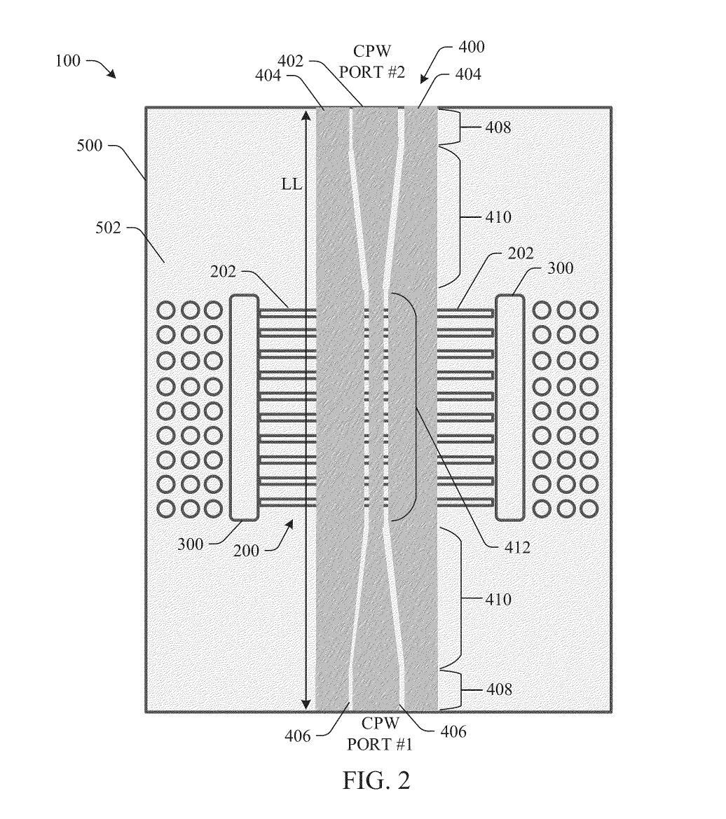

FIG. 2 is a top view of an innovative microfluidic bio-detection chip (hereinafter “microfluidic chip”) 100 in accordance with an aspect of the innovation. The microfluidic chip 100 includes a microchannel region 200 having microfluidic-channels (hereinafter “channels”) 202, reservoirs 300, and a coplanar-waveguide (CPW) transmission line (hereinafter “CPW transmission line”) 400. The microfluidic chip 100 may be fabricated on a dielectric substrate 500, which may be comprised of a polymer, such as but not limited to polydimethylsiloxane (PDMS). The channels 202 and reservoirs 300 are located below a top surface 502 (e.g., sub-surface) of the substrate 500. The channels 202 and are filled with aqueous solutions supplied by capillary action from the reservoirs 300. An object of the microfluidic chip is to make the channels 202 and reservoirs 300 as compact as practical to accommodate minimum sample volume. For example, in one embodiment the channels 202 may have a width of approximately 2 microns and a depth of approximately 2 microns.

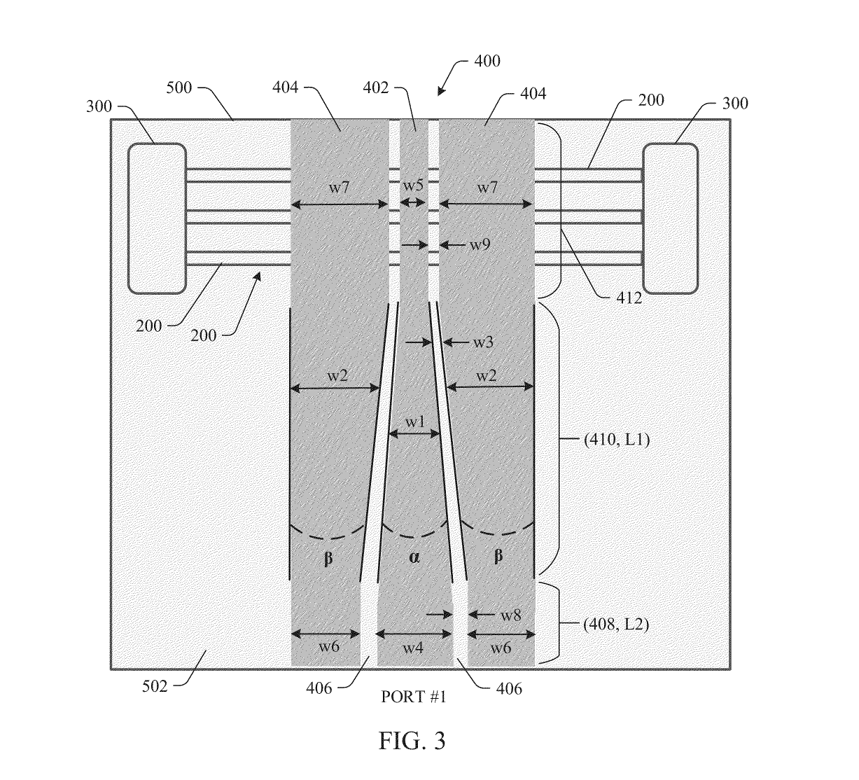

Referring to both FIGS. 2 and 3, the CPW transmission line 400 is comprised of a signal (center) conductor 402 and a ground or return (outer) conductor 404 disposed on the top surface 502 of the substrate 500 on each side of the signal conductor 402 such that the conductors 402, 404 are coplanar. A continuous gap 406 is defined between the signal conductor 402 and each of the ground conductors 404. As will be described further below, a width of the continuous gaps 406 changes or varies between regions or sections along a longitudinal length LL of the CPW transmission line 400.

The CPW transmission-line 400 further includes a pair of pad regions (or signal/ground pad sections) 408, a pair of taper (condenser) regions (or signal/ground taper sections) 410, and a microfluidic region (or signal/ground microfluidic sections) 412. The pair of pad regions 408 include a first CPW port and a second CPW port that provide a coupling to a vector network analyzer (VNA) through probes known as ground-signal-ground (GSG) probes. In one example embodiment, the pair of pad regions 408 may be configured for coupling to 100-μm pitch GSG probes. In addition, the characteristic impedance (e.g., 50Ω) of the CPW transmission line 400 at the pair pad regions 408, in one example embodiment, may be configured to match an impedance of the GSG probes.

The pair of taper regions 410 connect the pair of pad regions 408 to opposite ends of the microfluidic region 412. Referring to FIG. 3, the taper region 410 is configured to provide a continuous, linear reduction of the CPW transmission line 400 from the pad region 408 to the microfluidic region 412. In other words, a width w1 of the signal conductor 402 in the taper region 410 decreases from the pad region 408 to the microfluidic region 412 (or increases from the microfluidic region 412 to the pad region 408). This decrease (increase) is defined as a signal conductor taper angle α that can range from 2-6 degrees. Thus, as will be described further below, a distance between the pair of gaps 406 is less at the microfluidic region 412 than at the pad region 408. The continuous taper facilitates a continuous impedance along the taper region 410 to maintain matching characteristics of the CPW transmission line 400.

Similarly, a width w2 of the ground conductors 404 also varies in the taper region 410 between the pad region 408 and the microfluidic region 412. Specifically, the width w2 increases from the pad region 408 to the microfluidic region 412 (or decreases from the microfluidic region 412 to the pad region 408), which is defined by a ground conductor taper angle β. The ground conductor taper angle β, however, is not equal to the signal conductor taper angle α. In other words, the width w2 of the ground conductors increases/decreases at an angle that is different than the angle of increase/decrease of the width w1 of the signal conductor 402. As a result, a width w3 of each continuous gap 406 decreases from the pad region 408 to the microfluidic region 412 (or increases from the microfluidic region 412 to the pad region 408).

Still referring to FIG. 3, having a length L1 of the taper region 410 to be greater than a length other the regions of the CPW transmission line 400 further facilitates the continuous impedance between the pad region 408 and the microfluidic region 412. For example, in one embodiment, the length L1 of the taper region 410 may be 500 μm, whereas a length L2 of the pad region 408 may be 100 μm. It is to be understood that both lengths L1, L2 may vary based on parameters, such as but not limited to the type of application the microfluidic chip 100 may be used in.

The microfluidic region 412 is a narrow section that runs directly over a top of the channels 202. The microfluidic region 412 is narrower than the pad regions 408, which maximizes the interaction between electric fields of the CPW transmission line 400 and the channels 202. This primarily occurs in the gaps 406 between the signal conductor 402 and the two ground conductors 404.

More specifically, the signal conductor 402 has a width w4 (e.g., 50-100 μm) in the pad region 408 and a width w5 (e.g., 14-28 μm) in the microfluidic region 412 where w4 is greater than w5. More specifically, the width w5 of the signal microfluidic section 412 is less than half of the width w4 of the signal pad section 408. The ground conductors 404 have a width w6 (e.g., 124-152 μm) in the pad region 408 and a width w7 (e.g., 162-171 μm) in the microfluidic region 412 where w6 is less than w7. As mentioned above, the width w1, w2 of both the signal and ground conductors 402, 404 respectively varies between the pad region 408 and the microfluidic region 412. It is to be understood that width of the signal and ground conductors 402, 404 in the regions 408, 410, 412 may be any value (including values outside the example ranges provided above) based on parameters, such as but not limited to the type of application the microfluidic chip 100 may be used in.

In addition, the continuous gaps 406 have a width w8 in the pad region 408 and a width w9 in the microfluidic region 412 where w8 is greater than w9. For example, in one embodiment the width w8 in the pad region 408 may be as little as 3 μm to as much as 6 μm and the width w9 of the gap 406 in the microfluidic region 412 may be a little as 2 μm and as much as 4 μm. Thus, in this example embodiment, since the width w3 of the continuous gaps 406 varies in the taper region 410, the width w3 of the continuous gap 406 may decrease from 3 μm at the pad region 408 to 2 μm at the microfluidic region 412 or from 6 μm at the pad region to 4 μm at the microfluidic region 412. It is to be understood that width of the gaps 406 in the regions 408, 410, 412 may be any value based on parameters, such as but not limited to the type of application the microfluidic chip 100 may be used in.

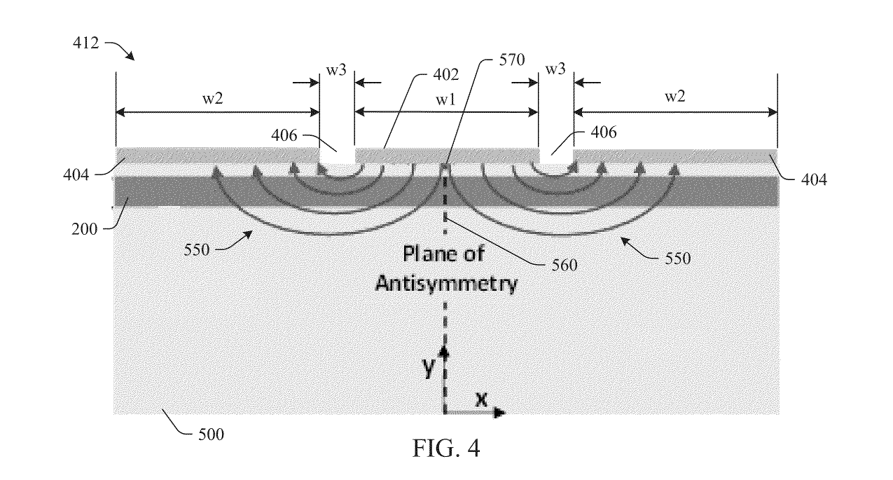

FIG. 4 is a cross-sectional view of the microfluidic chip 100 through the microfluidic region 412. FIG. 4 also illustrates electric fields (lines of force) 550 in the gaps 406 between the signal and ground conductors 402, 404 and extend down to a depth comparable to the gap 406 width. As illustrated above, the microfluidic region 412 is where the width w1 of the signal conductor 402 and the gaps 406 are at their narrowest. This configuration meets two conditions: (1) to create a high coupling between the electric field 550 in the CPW transmission line 400 and the channels 202, and (2) to maintain as closely as possible a constant impedance (e.g., 50 ohms) in the heterogeneous substrate medium 500 and the channels 202.

Regarding the electric fields 550, assuming that the CPW transmission line 400 is propagating only its “even” mode, whereby a plane of antisymmetry 560 exists through a geometric center 570 of the CPW transmission line 400, then for an x-y coordinate system as shown in FIG. 4, the antisymmetry can be stated as E(x)=−E(−x), independent of y, where E is the instantaneous electric-field vector. This is important because of the presence of the channels 202, which break up the homogeneity of the substrate 500.

The effect of the channels 202 can be represented by their geometry and by the dielectric constant of the solution filling them. Assuming the channels 202 are uniformly filled, the “effective” dielectric ∈_{S} of the substrate in the presence of the microfluidic channels 202 will display symmetry with respect to the same plane, i.e., ∈_{S}(x)=∈_{S}(−x). And because ∈_{S} is a scalar quantity, its symmetry is consistent with the antisymmetry of the electric field 550 of the CPW transmission line 400 even mode. In other words, the microchannel region will not scatter the even mode, or transform it to other possible propagating modes, such as the odd mode. The microchannel region 200, however, can readily induce the absorption of propagating radiation, especially with the channels 202 full of aqueous solution.

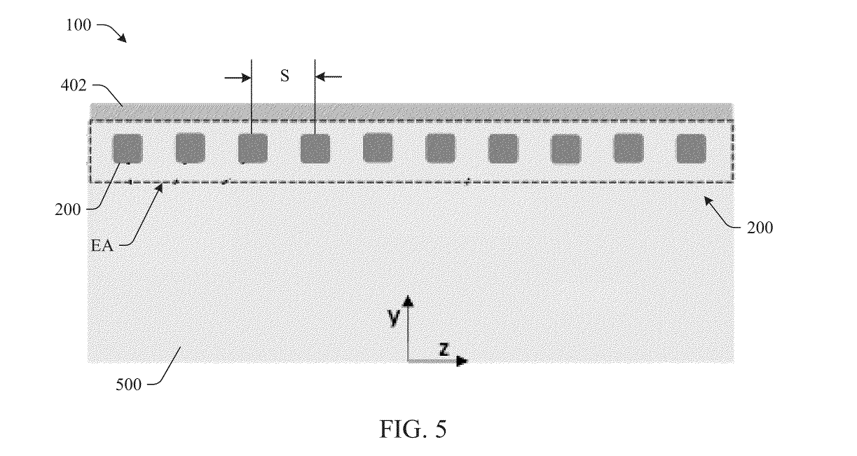

An Effective-Media Analysis can be performed to predict and confirm this effect. FIG. 5 is a side view of the microchannel region 200 in a cut taken vertically down an axis of propagation and through a center of the signal conductor 402. As illustrated in FIG. 5, the channels 202 are uniformly spaced and nominally having a center-to-center separation S approximately equal to twice their width. Combined with the lateral symmetry described above, there is a translational symmetry down the propagating axis. If multiple channels 202 are considered at the same time, over a length scale much less than a propagating wavelength, then ∈_{S} can be estimated using effective-media theory. For example, an effective area EA in FIG. 5 contains 10 translational periods of the channels 202. So assuming the period is 4 micron, there is an effective length of 40 microns, which corresponds to an effective wavelength of λ_{eff}=40×(Re{∈ₛ})^{1/2} [um]. If the channels 202 were pure PDMS, for which ∈ᵣ=2.67, then ∈_{S}=∈ᵣ=2.67, and λ_{eff}≈65 um, for which the frequency is f=c/λ_{eff}=4.6 THz.

An aqueous solution will change ∈_{S}, and to predict that effect the Maxwell-Garnett model of effective-media theory is utilized where the expression for the effective dielectric constant is:

$${ɛ}_{eff} = {ɛ}_{B} + \frac{\frac{1}{3} {f}_{I} ( {ɛ}_{I} - {ɛ}_{B} ) {\sum }_{i = 1}^{3} \frac{{ɛ}_{B}}{{ɛ}_{B} + {N}_{i} ( {ɛ}_{I} - {ɛ}_{B} )}}{1 - \frac{1}{3} {f}_{I} ( {ɛ}_{I} - {ɛ}_{B} ) {\sum }_{i = 1}^{3} \frac{{N}_{i}}{{ɛ}_{B} + {N}_{i} ( {ɛ}_{I} - {ɛ}_{B} )}} \qquad ( 1 )$$

where ∈_{B} and ∈_{I} are the dielectric functions of the background host and inclusion, respectively, f_{I} is the volumetric fill factor of the inclusion, and Ni is a depolarization factor of the inclusion taken along three orthogonal axes. The dielectric function of PDMS has at least for frequencies below 100 GHz, ∈_{B} is primarily real. For example, at 77 GHz, ∈_{B}≈2.67, and tan δ≈0.04, where tan δ is the dielectric loss tangent defined by tan δ≡Im{∈}/Re{∈} in non-conducting material. Henceforth, we will assume ∈_{B} is real and =2.67.

The depolarization factor requires further investigation. For example, an infinite cylinder whose axis is aligned along the i=1 (x axis) of FIG. 4, N₁≈0, N₂≈½, and N₃≈½. This is a good first approximation based on the following reasoning. A more exact estimate would be based on the distance over which the cylinder lies in significant lateral electric fields in FIG. 4. This is roughly the twice the sum of the center conductor plus two gap widths. And the ratio of this to the channel width is so much greater than unity that the cylinder can be considered infinite with respect to the propagating (z) direction. This simplifies Eq. (1) to the form:

$${ɛ}_{eff} = {ɛ}_{B} + \frac{\frac{4}{3} {f}_{I} ( {ɛ}_{I} - {ɛ}_{B} ) \frac{{ɛ}_{B}}{2 {ɛ}_{B} + ( {ɛ}_{I} - {ɛ}_{B} )}}{1 - \frac{2}{3} {f}_{I} ( {ɛ}_{I} - {ɛ}_{B} ) \frac{1}{2 {ɛ}_{B} + ( {ɛ}_{I} - {ɛ}_{B} )}} \qquad ( 2 )$$

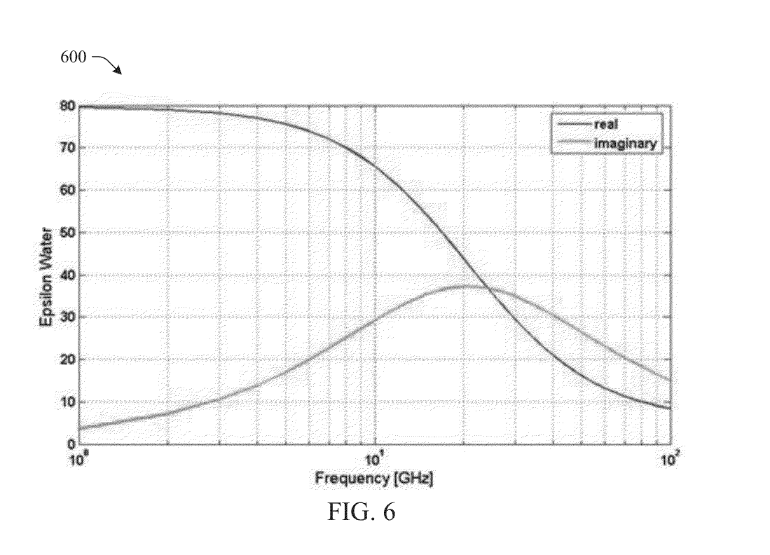

Next, an estimate of the dielectric function, ∈_{I}, of the aqueous solution is required. Assuming that like most biological solutions, the pH is close to neutral and the salinity is low. Under those conditions, the dielectric function of an aqueous solution from RF-to-THz frequencies is adequately described by the double-Debye model:

$${ɛ}_{w} = {ɛ}_{∞} + \frac{{ɛ}_{0} - {ɛ}_{1}}{1 - j ω ( \frac{v}{{v}_{1}} )} + \frac{{ɛ}_{1} - {ɛ}_{∞}}{1 - j ω ( \frac{v}{{v}_{2}} )} \qquad ( 3 )$$

where ∈₀ is the dc value of 79.7, ∈_{∞} is the infinite-frequency value of 3.37, and ∈₁ is the intermediate value of 5.35. The other key parameters in this model are the relaxation frequencies, v₁=20.7 GHz, and v₂=692 GHz. These are inversely related to characteristic relaxation times, τ₁=7.7 ps, and τ₂=0.23 ps. The resulting curves for the real and imaginary parts of ∈_{w} are plotted in the graph 600 shown in FIG. 6.

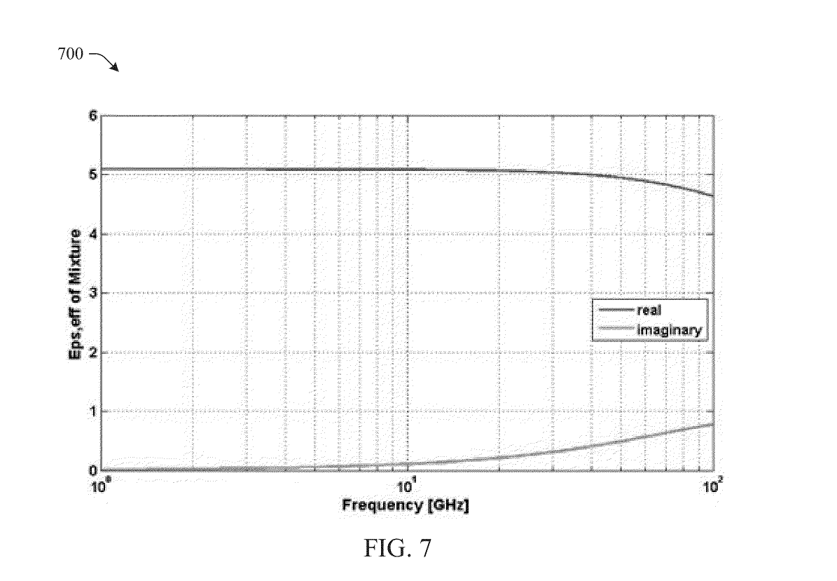

To complete the effective-medium analysis of the microfluidic region 412, a fill factor of the channels 202 relative to the substrate 500 must be estimated. The estimation entails both volumetric and electric-field-strength effects. However, referring to FIGS. 4 and 5, it appears that f_{I}=0.5 (i.e., 50%) is a reasonable estimation. The resulting real and imaginary parts of Eq. (2) are plotted in the graph 700 shown in FIG. 7. There is a dramatic change in both parts of the mixture dielectric function compared to the pure aqueous solution illustrated in FIG. 6. Both are decreased dramatically in magnitude, and the imaginary part stays well below the real part over the range of 1-100 GHz. This suggests that there is a great reduction in the host aqueous attenuation in the microfluidic region 412 compared to the uniformly-filled region. The attenuation coefficient from the dielectric function is calculated to gain a better understanding.

Whether in a homogeneous or composite medium, the attenuation coefficient cc can be calculated from the real and imaginary parts of the effective dielectric constant by introduction of the complex refractive index, n=(∈)^{1/2}. Then

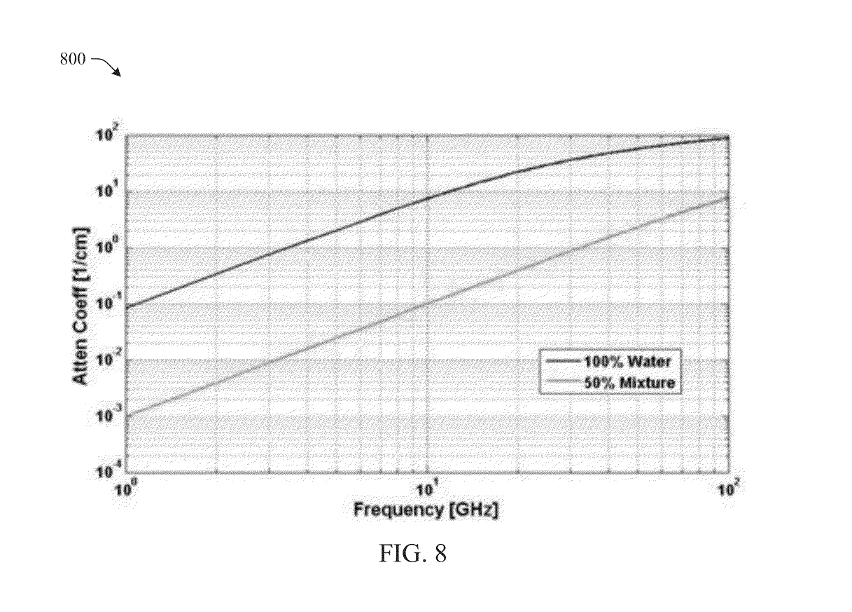

α=2*Imag{n}*k (4)

where k=ω/c is the propagation constant where c is the speed of light in vacuum. A plot 800 of α is shown in FIG. 8 for the microchannel region 200 (50% fill factor) and for 100% water. The attenuation of the 100%-water is much stronger than that of the microchannel region 200, by about a factor of 100 at 1 GHz, decreasing to a factor of 10 at 100 GHz. According to Beer's law of optics, the radiation intensity of a propagating wave in an attenuating medium depends on distance of travel z as I=I₀ exp(−α·z). A similar dependence occurs in guided waves like the even mode of the CPW transmission line 400 in the microfluidic region 412. A characteristic attenuation length z=L is that which makes the exponent term exp(−1)=0.37 (−4.3 dB). Substitution into Beer's law then yields L=1/α.

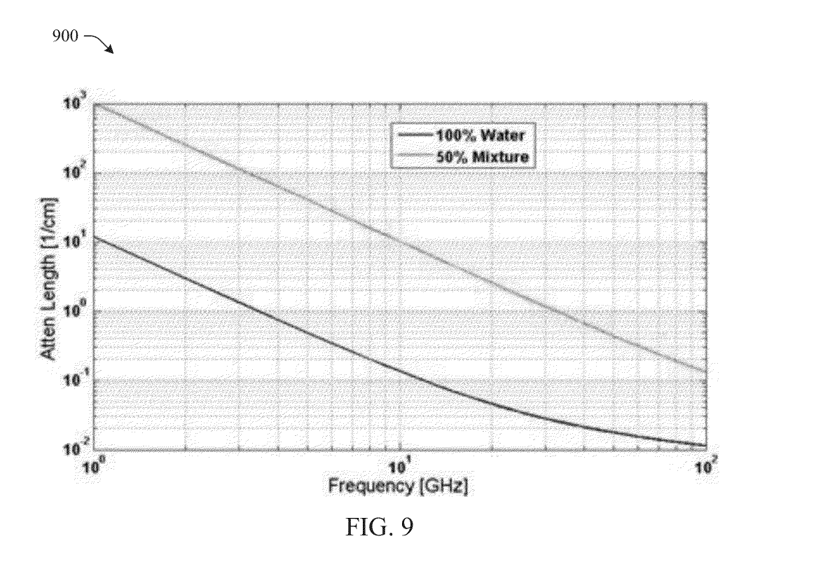

The attenuation length is plotted in the graph 900 shown in FIG. 9. This plot serves as a design guide for the microchannel region 200. In order to enhance the detectability of viruses in the microchannel region, we want to make it as long as possible with as many channels 202 as possible, constrained only by the background attenuation of the water, and by the availability of the aqueous solution.

Given ample availability, L then serves as a maximum length for which the background attenuation will be −4.3 dB, which is an acceptable loss (“insertion loss” in microwave-engineering jargon). However, as seen in FIG. 9, this L is a strong function of frequency because of the strong dispersion of the water dielectric function of Eq. 3. As an example, take the HPV-16 T=1 viral protein predicted by Indiana University to display a self-oscillation frequency of approximately 16 GHz. According to FIG. 9, the attenuation length of the microchannel region 200 at this frequency is approximately 3 cm. So a cm-length microchannel region 200 will suffer negligible background attenuation.

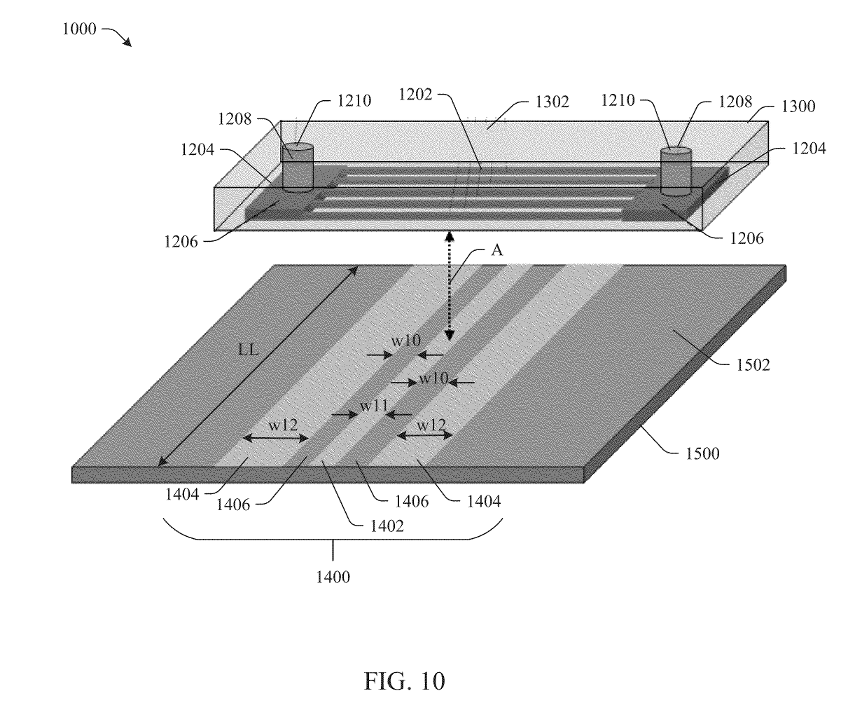

FIG. 10 illustrates another example embodiment of an innovative microfluidic bio-detection chip (hereinafter “microfluidic chip”) 1000 having a substrate-superstrate (SS) structure in accordance with an aspect of the innovation. The microfluidic chip 1000 includes a microchannel region 1200 disposed in a superstrate 1300 (i.e., below a top surface 1302 of the superstrate 1300), a coplanar waveguide (CPW) transmission line 1400 disposed on a substrate 1500 (i.e., on a top surface 1502 of the substrate 1500). This configuration separates the CPW transmission-line patterning from the microfluidic fabrication, the former occurring on the substrate and the latter on the superstrate. The superstrate 1300 may be made from made from a dielectric polymer, such as but not limited to polydimethylsiloxane (PDMS). The substrate 1500 may be made from an insulating material, such as but not limited to quartz, high-resistivity silicon, etc. The channels 1202 have an open channel configuration in the superstrate 1300 and may be fabricated by a low-cost stamping technique. The CPW transmission line 1400 may be fabricated on the insulating substrate 1500 by planar processing; e.g., photolithography, metallization, etc. The superstrate 1300 and the substrate 1500 can then be joined as illustrated by the double arrow A in FIG. 10 by a technique that provides a hermetic seal (e.g., plasma bonding technique). Thus, the superstrate 1300 is disposed on the top surface 1502 of the substrate when the two are joined.

The microchannel region 1200 includes microfluidic channels 1202, reservoirs 1204 having a top surface 1206, and fill ports 1208. The channels 1202 run substantially parallel between the reservoirs 1204 where the reservoirs 1204 supply an aqueous solution to the channels 1202. The fill ports 1208 extend upward from the top surface 1206 of each reservoir 1204 and are exposed from the superstrate 1300 such that the aqueous solution can be provided to the reservoirs 1204 via the fill ports 1208. Thus, a top surface 1210 of each fill port 1208 may be flush with the top surface 1302 of the superstrate 1300 or may extend above the top surface 1302 of the superstrate 1300.

Still referring to FIG. 10, the CPW transmission line 1400 is comprised of a signal (center) conductor 1402 and a ground or return (outer) conductor 1404 disposed on the top surface 1502 of the substrate 1500 on each side of the signal conductor 1402 such that the conductors 1402, 1404 are coplanar. A continuous gap 1406 is defined between the signal conductor 1402 and each of the ground conductors 1404. In one example embodiment, the continuous gaps 1406 may have a constant width w10 and thus, a width w11 of the center conductor 1402 and a width w12 of the outer conductors may have a constant width along a longitudinal length LL of the CPW transmission line 1400. In another example embodiment, the width w10 of the continuous gaps 1406 may vary as described above. Thus, the widths w11, w12 of the center conductor and outside conductors 1402, 1404 may vary as described above.

The dielectric environment of the CPW transmission line 1400 is different in the SS structure than in the substrate only structure described above. This is quantified by the effective dielectric constant ∈_{eff}. Most electromagnetic computer-aided design (CAD) tools for coplanar transmission lines assume a half-space of air above the substrate, so they work very well for the launching regions (the end of the transmission lines shown in FIG. 10). To compensate for the transition from substrate only structure to the SS structure, one aspect of the CPW transmission line 1400 leads to a simplification in the analysis. The separation between “hot” electrode and the ground plane (or planes) is necessarily of the order of the microfluidic channel width, which may be approximately a few microns. In some example embodiments, however, the insulating substrate 1400 and the superstrate 1300 are much thicker than this, approximately 500 microns. Thus, both can be treated as dielectric half-spaces. Then ∈_{eff} in the SS structure is approximately given by the arithmetic mean:

∈_{ff,SS}≈(∈_{sub}+∈ₛᵤₚ)/2 (5)

and the fundamental mode velocity is given by

vₛₛ≈c/(∈_{eff})^{1/2} (6)

where c is the speed of light in vacuum.

The critical design issue in the SS structure is to make the transmission-line characteristic impedance Z_{0,SS}, match the characteristic impedance in the substrate-only region, Z₀ (e.g., approximately 50 Ohms). An equivalent-circuit representation of lossless transmission line is utilized whereby the electromagnetic-wave behavior can be represented by a series specific inductance L′ (inductance per unit length) and a shunt specific capacitance C′ (capacitance per unit length). With these circuit parameters the following can be written:

v≈1/(L′C′)^{1/2} (7)

Zₒ≈(L′/C′)^{1/2} (8)

These relationships are exact for TEM-type transmission lines (e.g., coaxial), but are acceptably accurate for quasi-TEM lines such as the coplanar types used for the present invention.

The dimensions of the coplanar line for which Z_{0,SS}≈50 Ohms can be found by an equivalence method which assumes a fictitious coplanar line having the same dimensions as the SS line but on an insulating half-space of the substrate alone. Because the metal dimensions are the same and there are no magnetic materials anywhere, the specific inductance of the fictitious line will be the same as for the substrate-superstrate line, L′_{SS}. By taking the ratio of Eq. (8) to Eq. (7), C′ is canceled and get the useful relationship:

L′=Z₀/v (9)

We set this in turn to the specific inductance of the actual substrate-superstrate structure and the fictitious structure, getting the relation:

Z_{0,F}/v_{F}=Z_{0,SS}/vₛₛ (10)

Although less accurate than Eq. (5), we can approximate the effective dielectric constant of the fictitious line as

∈_{eff,F}≈(∈_{sub}+1)/2 (11)

and the fundamental mode velocity as

v_{ss,F}≈c/(∈_{eff,F})^{1/2} (12)

Substitution of Eq. (6) and Eq. (12) into Eq. (10) then yields a design expression,

Z_{0,F}=Z_{0,SS}[(∈_{sub}+∈ₛᵤₚ)/(∈_{sub}+1)]^{1/2}≡50·[(∈_{sub}+∈ₛᵤₚ)/(∈_{sub}+1)]^{1/2} (13)

Knowing ∈_{sub} and ∈ₛᵤₚ, a transmission-line design tool can be used to get the dimensions of the fictitious line, and those will be the same dimensions that achieve 50 Ohm in the SS structure.

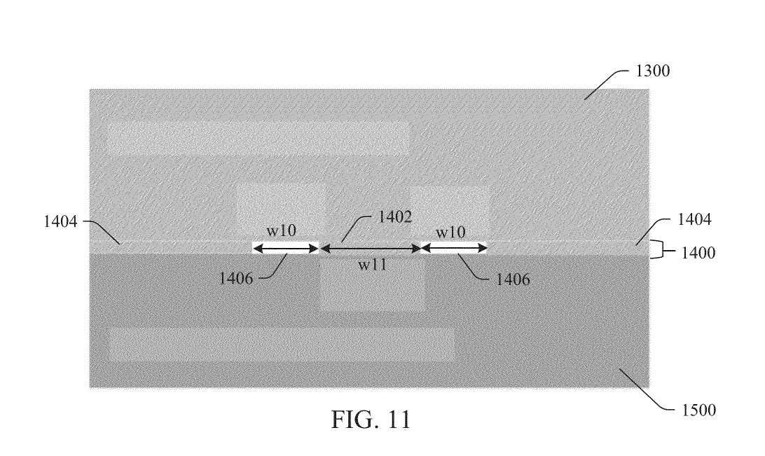

In an example embodiment, FIG. 11 illustrates an end-view of the CPW transmission line 1400 on a substrate 1500 of fused quartz and a superstrate 1300 of PDMS disposed over the CPW transmission line 1400. In the GHz-to-THz region the relative dielectric constant for fused quartz is approximately ∈ᵣ=3.80 and for PDMS ∈ᵣ=2.67. Eq. (13) then evaluates to Z_{0,F}=50*1.16=58.0 Ohm. The dimensions of the microfluidic channels in the superstrate 1300 are assumed to be large enough for ease-of-fabrication, but small enough to be filled by a very low quantity of biological sample. One example dimension can be 4×4 um. The width w10 of the continuous gap 1406 in the CPW line is then chosen to provide good interaction between the electric field lines and the channels. For example, one value of w10 may be 4 um. Then the width w11 of the center conductor 1402 can be determined by trial-and-error using a standard microwave transmission-line CAD program under the constraint that Z_{0,F}=58.0 Ohm. Using the CAD program TX-LINE, it is found that w11=25 um. It is to be understood that the example embodiment illustrated in FIG. 11 is for illustrative purposes only and is not intended to limit the scope of the innovation.

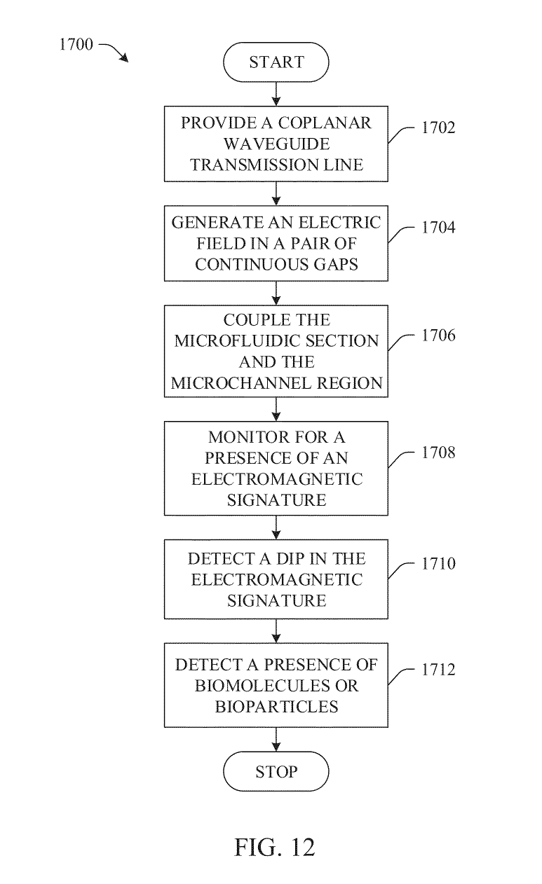

Referring to FIG. 12, a method of detecting a virus in a biosample is illustrated in a block diagram 1700 in accordance with an aspect of the innovation. At 1702, a coplanar waveguide transmission line, which includes a pad section, a taper section, and a microfluidic section, where the microfluidic section is disposed above the microchannel region, as disclosed herein. At 1704, an electric field is generated in the pair of continuous gaps defined in the coplanar waveguide transmission line, as disclosed herein. At 1706, the electric field provides a coupling between the microfluidic section and the microchannel region. At 1708, a presence of an electromagnetic signature in a biosample is monitored. At 1710, a dip in the electromagnetic signature is detected. At 1712, biomolecules and bioparticles are detected.

As mentioned above, the radiative interrogation of the innovative microfluidic chip 100 can be done in such a way that a unique signature appears in the transmittance vs. frequency graph illustrated in FIG. 1. This can be done with high resolution (<1 GHz, if needed) and high dynamic range (up to 80 dB). Thus, the signature can be detected very quickly and sensitively in the presence of instrumental noise and background attenuation of the aqueous solution harboring the biomolecule or bioparticle of interest. This is one of the advantages of the innovation, which is data acquisition time. As mentioned above, a limitation of the state-of-the-art in bioparticle sensing as applied to viruses is the measurement time, which is presently at least one hour. The innovation disclosed herein significantly decreases the measurement time down to as little as one minute.

Another advantage is autonomy. With a signature-to-noise ratio as shown in FIG. 1, the signature can be identified by various signature identification software, some based on cross correlation algorithms. This will increase the probability of detection and decrease the false-alarm rate to the extent that the presence of the biomolecule or bioparticle of interest can be established by a computer instead of the human operator. This will reduce the training and experience required of operators, allowing them to concentrate on the sample-collection task and carry out much more sampling in a given time than if data analysis was required.

Another advantage is that the innovation is safer than existing technology from a biological threat standpoint. The innovation allows for the detection of molecules or particles at their in-situ concentration levels, expected to be parts-per-billion at locations where human-health is affected. On the other hand, existing technology, especially PCR, must multiply the genomic matter roughly one-million fold, thereby creating concentrations that could be lethal in the event of a leakage. Further, the innovation can analyze samples inside polyethylene sample bags without exposing workers to the agents.

One market for the innovation could be the Departments of Defense and Homeland Security. Other applications and uses can include the monitoring of live viruses in public places and likely areas of proliferation, such as hospitals, nursing homes, airports, train stations, etc. The application would likely entail swabbing certain items such as bathroom door handles, check-in counter tops, etc. that are subject to continuous human handling. The swab contents would then be transferred to the reservoir of the microfluidic chip, and the chip would be loaded into the RF analyzer for interrogation. Other sample collection devices could include air and water filters. Many different viral species could be detected, with sensitivity levels that depend on the strength of each vibrational signature. In addition, the innovation can be easily modified to accommodate different applications, uses, sample collection considerations, etc.

What has been described above includes examples of the innovation. It is, of course, not possible to describe every conceivable combination of components or methodologies for purposes of describing the subject innovation, but one of ordinary skill in the art may recognize that many further combinations and permutations of the innovation are possible. Accordingly, the innovation is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A coplanar waveguide transmission line for use in detecting biomolecules and bioparticles comprising:
a dielectric substrate;
a signal conductor disposed on a top surface of the dielectric substrate;
a ground conductor disposed on the top surface of the dielectric substrate on each side of the signal conductor;
a continuous gap defined between the signal conductor and each of the ground conductors;
a plurality of micro-channels disposed below a top surface of the dielectric substrate; and
a plurality of reservoirs disposed below the top surface of the substrate, wherein the plurality of reservoirs supply an aqueous solution to the micro-channels.

2. The coplanar waveguide transmission line of claim 1, wherein the signal conductor includes a signal pad section, a signal taper section, and a signal microfluidic section and wherein a width of the signal microfluidic section is less than half a width of the signal pad section.

3. The coplanar waveguide transmission line of claim 2, wherein the ground conductor includes a ground pad section, a ground taper section and a ground microfluidic section and wherein a width of the ground microfluidic section is greater than a width of the ground pad section.

4. The coplanar waveguide transmission line of claim 3, wherein a width of the signal taper section decreases from the signal pad region to the signal microfluidic section and wherein a width of the ground taper section increases from the ground pad section to the microfluidic section.

5. The coplanar waveguide transmission line of claim 4, wherein the signal taper section includes a signal taper angle and the ground taper section includes a ground taper angle and wherein the signal taper angle is not equal to the ground taper angle.

6. The coplanar waveguide transmission line of claim 5, wherein a width of the continuous gap between the signal taper section and the ground taper section decreases from the signal and ground pad sections to the signal and ground microfluidic sections.

7. The coplanar waveguide transmission line of claim 6, wherein a width of the continuous gap between the signal microfluidic region and the ground microfluidic regions is less than a width of the continuous gap between the signal pad section and the ground pad sections.

8. The coplanar waveguide transmission line of claim 2, wherein the signal microfluidic section and the ground microfluidic section are located over the plurality of micro-channels and wherein an electric field is generated between the signal conductor and the ground conductors that provides a coupling between the signal and ground microfluidic sections and the plurality of channels to thereby detect the biomolecules and bioparticles.

9. The coplanar waveguide transmission line of claim 1, wherein an impedance along the longitudinal length of the signal conductor and each ground conductor remains approximately consistent to thereby match an impedance of ground-signal-ground probes.

10. A microfluidic bio-detection chip comprising:
a dielectric substrate;
a coplanar waveguide transmission line disposed on a top surface of the dielectric substrate, the coplanar waveguide transmission line including:
a pad region;
a microfluidic region; and
a taper region connecting the pad region to the microfluidic region;
a pair of continuous gaps extending along the coplanar waveguide transmission line;
a microchannel region disposed below a top surface of the dielectric substrate; and
a plurality of reservoirs disposed below the top surface of the substrate, wherein the plurality of reservoirs supply an aqueous solution to the microchannel region.

11. The microfluidic bio-detection chip of claim 10, wherein the coplanar waveguide transmission line includes a signal conductor and wherein a width of the microfluidic region of the signal conductor is less than half a width of the pad region of the signal conductor.

12. The microfluidic bio-detection chip of claim 10, wherein the coplanar waveguide transmission line includes a ground conductor situated on each side of the signal conductor and wherein a width of the microfluidic region of the ground conductor is greater than a width of the pad region of the ground conductor.

13. The microfluidic bio-detection chip of claim 10, wherein an impedance is approximately consistent along the pad region, the taper region, and the microfluidic region to thereby match an impedance of ground-signal-ground probes.

14. The microfluidic bio-detection chip of claim 10, wherein a width of the pair of continuous gaps in the microfluidic region is less than the width of the pair of continuous gaps in the pad region.

15. The microfluidic bio-detection chip of claim 14, wherein the width of the continuous gaps in the taper region decreases from the pad region to the microfluidic region.

16. The microfluidic bio-detection chip of claim 10, wherein the microfluidic region is disposed over the microchannel region.

17. The microfluidic bio-detection chip of claim 16, wherein electric filed lines are generated in the pair of continuous gaps and extend toward the microchannel region thereby providing a coupling between the microfluidic region and the microchannel region to thereby detect a presence of biomolecules and bioparticles.

18. A microfluidic bio-detection chip comprising:
a dielectric substrate;
a coplanar waveguide transmission line disposed on a top surface of the dielectric substrate;
a dielectric superstrate disposed on a top surface of the dielectric substrate; and
a microchannel region disposed below a top surface of the dielectric superstrate.

19. The microfluidic bio-detection chip of claim 18, wherein the microchannel region includes a plurality of microchannels, a plurality of reservoirs, and a fill port disposed on a top surface of each of the plurality of reservoirs.

20. The microfluidic bio-detection chip of claim 19, wherein the fill port extends from the top surface of each of the plurality of reservoirs toward the top surface of the superstrate such that a top surface of the fill port is flush with the top surface of the superstrate.
